(19) United States
(12) Patent Application Publication
(10) Pub. No.: US 2012/0015048 A1
(43) Pub. Date: Jan. 19, 2012

(54) SKIN CARE EMULSION COMPOSITION

(76) Inventors: Prithwiraj Maitra, Randolph, NJ (US); Anna Prilutsky, Princeton, NJ (US); Ying Sun, Belle Mead, NJ (US)

(21) Appl. No.: 13/178,899

(22) Filed: Jul. 8, 2011

Related U.S. Application Data
(60) Provisional application No. 61/362,433, filed on Jul. 8, 2010.

(51) Int. Cl. A61K 8/92; A61Q 19/00

(52) U.S. Cl. 424/638; 514/772.3

(57) ABSTRACT

In one aspect, the invention relates to a method of making an emulsion composition. The method includes forming a water-in-oil emulsion and adding a galvanic particulate to the water-in-oil emulsion. In another aspect, a water-in oil emulsion is provided. The water-in-oil emulsion includes a water phase emulsified in a continuous oil phase; and a galvanic particulate. The oil-in water emulsion has a yield stress of at least about 20 Pascals (Pa).

### FIELD OF THE INVENTION

The present invention relates to skin care emulsion compositions comprising a galvanic particulate and, in particular, skin care emulsion compositions comprising a galvanic particulate.

### BACKGROUND OF THE INVENTION

It is often desirable to include ingredients in skin care compositions to provide one of various benefits to the skin. However, certain active ingredients are susceptible to chemical degradation or other forms of inactivation by water. This water-susceptibility is highly unfortunate, since water is a very desirable medium in which to formulate skin care compositions. If one is forced to exclude water from the skin-care composition, then large concentrations of oily, expensive, and/or volatile materials are required, thus causing potential difficulties in aesthetics, cost, flammability, and the like.

For example the Applicants have found that galvanic particulates, as described in WO 2009/045720 and US 2007/0060862, are, in certain cases susceptible to potential stability problems and possible reduced activity when combined with water into an emulsion composition.

Applicants have now discovered that premature degradation of water-susceptible galvanic particulates in a skin care formulation can be prevented by first forming a water-in-oil emulsion and then adding the galvanic particulate to the water-in-oil emulsion. In accordance with a preferred embodiment of the invention, Applicants have found that the rheology of the emulsion should be such that the yield stress is greater than about 20 Pascals (Pa).

### SUMMARY OF THE INVENTION

According to the present invention, a stable water-in oil emulsion containing a galvanic particulate is provided. The oil-in water emulsion preferably has a yield stress of at least about 20 Pa.

In another aspect, the invention relates to a method of making a stable emulsion composition by forming a water-in-oil emulsion and adding a galvanic particulate to the water-in-oil emulsion.

### DETAILED DESCRIPTION OF THE INVENTION

Unless defined otherwise, all technical and scientific terms used herein have the meaning commonly understood by one of ordinary skill in the art to which the invention pertains. All publications, patent applications, patents, and other references mentioned herein are incorporated by reference. Unless otherwise indicated, a percentage refers to a percentage by weight (i.e., % (W/W)).

As used herein, “cosmetically-acceptable” means suitable for use in topical contact with tissues (e.g., the skin) without undue toxicity, incompatibility, instability, irritation, allergic response, or the like. This term is not intended to limit the composition it describes as for use solely as a cosmetic (e.g., the composition may be used as a pharmaceutical).

As used herein, the terminology “safe and effective amount” means an amount sufficient to provide a desired benefit at a desired level, but low enough to avoid serious side effects.

As used herein, the terminology “treating” or “treatment” means alleviation or elimination of symptoms, cure, prevention, or inhibition of a human condition or disease, specifically of the skin.

As used herein, the terminology “Galvanic particulates” refers to a first conductive material that is in physical contact with a second conductive material, wherein both the first conductive material and the second conductive material are exposed on the surface of the galvanic particulate. By “particulate,” it is meant a finely divided material that is generally solid at room temperature and insoluble in either the water phase or the oil phase of an oil in water emulsion. In one embodiment, the galvanic particulates comprise the first conductive material and the second conductive material co-existing on the surface of individual galvanic particles. In one embodiment, the galvanic particulates comprise the first conductive material partially coated with the second conductive material. In another embodiment, the first conductive material is elemental zinc, and the second conductive material is elemental copper. In one embodiment, the galvanic particulates are produced by a coating method wherein the weight percentage of the second conductive material is from about 0.001% to about 20%, by weight, of the total weight of the particulate, such as from about 0.01% to about 10%, by weight, of the total weight of galvanic particulate. In one embodiment, the coating thickness of the second conductive material may vary from single atom up to hundreds of microns. In yet another embodiment, the surface of the galvanic particulate comprises from about 0.001 percent to about 99.99 percent such as from about 0.1 to about 99.9 percent of the second conductive material.

In one embodiment, the galvanic particulate comprises at least 90 percent, by weight, of conductive materials (e.g., the first conductive material and the second conductive material), such as at least 95 percent, by weight, or at least 99 percent, by weight, when a coating method is used for the production of the galvanic particulates.

Examples of combinations of first conductive materials and second conductive materials include (with a “/” sign representing an oxidized but essentially non-soluble form of the metal), but are not limited to, zinc-copper, zinc-copper/copper halide, zinc-copper/copper oxide, magnesium-copper, magnesium-copper/copper halide, zinc-silver, zinc-silver/silver oxide, zinc-silver/silver halide, zinc-silver/silver chloride, zinc-silver/silver bromide, zinc-silver/silver iodide, zinc-silver/silver fluoride, zinc-gold, zinc-carbon, magnesium-gold, magnesium-silver, magnesium-silver/silver oxide, magnesium-silver/silver halide, magnesium-silver/silver chloride, magnesium-silver/silver bromide, magnesium-silver/silver iodide, magnesium-silver/silver fluoride, magnesium-carbon, aluminum-copper, aluminum-gold, aluminum-silver, aluminum-silver/silver oxide, aluminum-silver/silver halide, aluminum-silver/silver chloride, aluminum-silver/silver bromide, aluminum-silver/silver iodide, aluminum-silver/silver fluoride, aluminum-carbon, copper-silver/silver halide, copper-silver/silver chloride, copper-silver/silver bromide, copper-silver/silver iodide, copper-silver/silver fluoride, iron-copper, iron-copper/copper oxide, copper-carbon iron-copper/copper halide, iron-silver, iron-silver/silver oxide, iron-silver/silver halide, iron-silver/silver chloride, iron-silver/silver bromide, iron-silver/silver iodide, iron-silver/silver fluoride, iron-gold, iron-conductive carbon, zinc-conductive carbon, copper-conductive carbon, magnesium-conductive carbon, and aluminum-carbon. When the first conductive and the second conductive materials are elemental metals (e.g., galvanic particulates of zinc-copper, zinc-silver, magnesium-copper, magnesium-silver, which are preferred galvanic particulates in the present invention), the first conductive metals are oxidizable metals (i.e., with high oxidation potential, or low reduction potential such as zinc and magnesium), and the second conductive metals are reducible metals (i.e., with low oxidation potential or high reduction potential such as copper and silver).

The first conductive material or second conductive material may also be alloys, particularly the first conductive material. Non-limiting examples of the alloys include alloys of zinc, iron, aluminum, magnesium, copper and manganese as the first conductive material and alloys of silver, copper, stainless steel and gold as second conductive material.

In another embodiment, the galvanic particulate can comprise a plurality of conductive materials or metals, namely, the number can be greater than 2 (binary) or 3 (tertiary). A non-limiting example of such a galvanic particulate can have the composition of magnesium-zinc-iron-copper-silver-gold in the form of multiple coatings or multiple conductive metal composite.

In one embodiment, the galvanic particulate, made of the first conductive material, is partially coated with several conductive materials, such as with a second and third conductive material. In a further embodiment, the particulate comprises at least 95 percent, by weight, of the first conductive material, the second conductive material, and the third conductive material. In one embodiment, the first conductive material is zinc, the second conductive material is copper, and the third conductive material is silver.

The galvanic particulates are water-susceptible particulates, i.e., when placed in contact with water, electrochemical reactions are induced that can used to provide benefits to the skin. By “water-susceptible particulate,” it is meant a particulate that is susceptible to partial or complete inactivation or degradation when in contact with water. Examples of water-susceptibility include, for example, partially or fully consumed electrical potential or reduced capacity to react electrochemically to generate galvanic electricity. In another embodiment, water-susceptibility includes reduced capacity to provide bioactivity. In another embodiment, water-susceptibility includes undesirable chemical reactivity of one or more ingredients in the composition. Additional effects of water-susceptibility may include undesirable color changes.

In one embodiment the particle density of the galvanic particulate is at least about 5 g/cc, such as at least about 6 g/cc. According to various embodiments of the invention, any of the following ranges of particle density may be suitable: 5 g/cc to about 9 g/cc, from about 6 g/cc to about 8 g/cc, from about 6 g/cc to about 9 g/cc, from about 7 g/cc, from about 9 g/cc, such as from about 7 g/cc, from about 8 g/cc. Accordingly, the skin care composition comprises up to about 10 weight percent galvanic particulates, for example up to about 5 weight percent galvanic particulates, such as from about 0.5 weight percent to about 4 weight percent, such as from about 1 weight percent to about 4 weight percent galvanic particulates.

In one embodiment, the galvanic particulates are fine enough that they can be suspended in the emulsion during storage. In a further embodiment, they are in flattened and/or elongated shapes. The advantages of flattened and elongated shapes of the galvanic particulates include a lower apparent density and, therefore, a better floating/suspending capability in the topical composition, as well as better coverage over the biological tissue, leading to a wider and/or deeper range of the galvanic current passing through the biological tissue (e.g., the skin or mucosa membrane). In one embodiment, the longest dimension of the galvanic particulates is at least twice (e.g., at least five times) the shortest dimension of such particulates.

In one embodiment, the average particle size of the galvanic particulates ranges from about 10 nanometers to about 500 micrometers, preferably from about 100 nanometers to about 100 micrometers, and more preferably form about 1 micrometer to about 50 micrometers. What is meant by the “particle size” is the maximum dimension measured in at least one direction of the particulates. The smaller the metal particles, the greater the galvanic reaction rate, hence more hydrogen peroxide can be generated.

In one embodiment, the galvanic particulates can be any shape, such as spherical, oblong, flake, rod, needle, and irregular shape. These particulates can be individual particles or aggregates, or as a coating on a metallic or non-metallic substrate or particles.

In one embodiment, the difference in the Standard Electrode Potentials (or simply, Standard Potentials) of the first conductive material and the second conductive material is at least about 0.1 volts, such as at least 0.2 volts. In one embodiment, the materials that make up the galvanic couple have a Standard Potential difference equal to or less than about 3 volts. For example, for a galvanic couple comprised of metallic zinc and copper, the Standard Potential of zinc is −0.763V (Zn/Zn2+), and the Standard Potential of copper is +0.337 (Cu/Cu2+), and the difference in Standard Potentials is therefore 1.100V for the zinc-copper galvanic couple. Similarly, for a magnesium-copper galvanic couple, the Standard Potential of magnesium (Mg/Mg2+) is −2.363V, and the difference in the Standard Potentials is therefore 2.700V. Additional examples of Standard Potential values of some materials suitable for use in galvanic particulates are: Ag/Ag+: +0.799V, Ag/AgCl/Cl−: 0.222V, and Pt/H2/H+: 0.000V. Pt may also be replaced by carbon or another conductive material. See, e.g., Physical Chemistry by Gordon M. Barrow, 4th Ed., McGraw-Hill Book Company, 1979, page 626.

In one embodiment, the first and second conductive electrodes are combined (e.g., the second conductive electrode is deposited to the first conductive electrode) by chemical, electrochemical, physical or mechanical process (such as electroless deposition, electric plating, vacuum vapor deposition, arc spray, sintering, compacting, pressing, extrusion, printing, and granulation) conductive metal ink (e.g., with polymeric binders), or other known metal coating or powder processing methods commonly used in powder metallurgy, electronics or medical device manufacturing processes, such as the methods described in the book Asm Handbook Volume 7: Powder Metal Technologies and Applications (Asm International Handbook Committee, edited by Peter W. Lee, 1998, pages 31-109, 311-320). In another embodiment, all the conductive electrodes are manufactured by chemical reduction processes (e.g., electroless deposition), sequentially or simultaneously, in the presence of reducing agent(s). Examples of reducing agents include phosphorous-containing reducing agents (e.g., a hypophosphite as described in U.S. Pat. Nos. 4,167,416 and 5,304,403), boron-containing reducing agents, and aldehyde- or keton-containing reducing agents such as sodium tetrahydridoborate (NaBH₄) (e.g., as described in US 2005/0175649).

In one embodiment, the second conductive electrode is deposited or coated onto the first conductive electrode by physical deposition, such as spray coating, plasma coating, conductive ink coating, screen printing, dip coating, metals bonding, bombarding particulates under high pressure-high temperature, fluid bed processing, or vacuum deposition.

In one embodiment, the coating method is based on displacement chemical reaction, namely, contacting particles of the first conductive material (e.g., metallic zinc particles) with a solution containing a dissolved salt of the second conductive material (e.g. copper acetate, copper lactate, copper gluconate, or silver nitrate). In a further embodiment, the method includes flowing the solution over particles of the first conductive material (e.g., zinc powder) or through a packed powder of the first conductive material. In one embodiment, the salt solution is an aqueous solution. In another embodiment, the solution is contains an organic solvent, such as an alcohol, a glycol, glycerin or other commonly used solvents in pharmaceutical production to regulate the deposition rate of the second conductive material onto the surfaces of the first conductive material particles, therefore controlling the activity of the galvanic particulates produced.

In another embodiment, the galvanic particulates of the present invention may also be coated with other materials to protect the first and second conductive materials from degradation during storage (e.g., oxidation degradation from oxygen and moisture), or to modulate the electrochemical reactions and to control the electric current generated when in use. Exemplary coating materials include inorganic or organic polymers, natural or synthetic polymers, biodegradable or bioabsorbable polymers, silica, glass, various metal oxides (e.g., oxide of zinc, aluminum, magnesium, or titanium) and other inorganic salts of low solubility (e.g., zinc phosphate). Coating methods are known in the art of metallic powder processing and metal pigment productions, such as those described in U.S. Pat. No. 5,964,936; U.S. Pat. No. 5,993,526; U.S. Pat. No. 7,172,812; US 20060042509A1 and US 20070172438.

In one embodiment, the galvanic particulates are stored in anhydrous form, e.g., as a dry powder or as an essentially anhydrous non-conducting organic solvent composition (e.g., dissolved in polyethylene glycol, propylene glycol, glycerin, liquid silicone, and/or alcohol). In another embodiment, the galvanic particulates are embedded into an anhydrous carrier (e.g., inside a polymer).

The inventors have now found that when galvanic particulates are formulated into certain emulsion compositions, water present in the emulsion can, perhaps through a similar mechanism generate premature “discharging” of the galvanic particulate. This can render the galvanic particulate less likely to deliver electrically-related benefits when later applied to the skin. The inventors have found that stable and efficacious emulsion compositions can now be made, by first forming a water-in-oil emulsion and then adding the water-susceptible galvanic particulate to the water-in-oil emulsion. The water-in oil emulsions preferably have a yield stress of at least about 20 Pascals (Pa).

The skin care compositions of the present invention may be any cosmetically-acceptable emulsion composition which includes a continuous oil phase and a discontinuous water phase emulsified in the oil phase. Compositions of the present invention further include a galvanic particulate that is suspended or dispersed in one or both of the oil and/or the water phase. The galvanic particulate is generally suspended or dispersed in the skin care composition by means of, e.g., steric (electrical) forces and/or by buoyancy forces due to a high sufficiently yield stress of the composition.

### Emulsion

As mentioned previously, skin care compositions of the present invention include a continuous oil phase and a discontinuous water phase emulsified in the oil phase. As one of ordinary skill in the art would understand, the continuous oil phase comprises the external phase of the emulsion, and may comprise an aggregation of one or more hydrophobic compounds. By “hydrophobic compound,” it is meant a compound that is generally insoluble in water and includes a hydrophobic moiety, such as one meeting one or more of the following three criteria: (a) has a carbon chain of at least six carbons in which none of the six carbons is a carbonyl carbon or has a hydrophilic moiety (defined below) bonded directly to it; (b) has two or more alkyl siloxy groups; or (c) has two or more oxypropylene groups in sequence. The hydrophobic moiety may include linear, cyclic, aromatic, saturated or unsaturated groups. The hydrophobic compound is in certain embodiments not amphiphilic and, as such, in this embodiment does not include hydrophilic moieties, such as anionic, cationic, zwitterionic, or nonionic groups, that are polar, including sulfate, sulfonate, carboxylate, phosphate, phosphonate, ammonium, including mono-, di-, and trialkylammonium species, pyridinium, imidazolinium, amidinium, poly(ethyleneiminium), ammonioalkylsulfonate, ammonioalkylcarboxylate, amphoacetate, and poly(ethyleneoxy)sulfonyl moieties. In certain embodiments, the hydrophobic compound does not include hydroxyl moieties.

The one or more hydrophobic compounds in the oil phase preferably include one or more oils. As used herein, “oils” means one or more hydrophobic compounds that have a melting point that is below 30° C. Suitable examples of compounds useful in or as the oil component include vegetable oils (glyceryl esters of fatty acids, triglycerides) and fatty esters. Specific non-limiting examples include, without limitation, esters such as isopropyl palmitate, isopropyl myristate, isononyl isonanoate (such as WICKENOL 151 available from Alzo Inc. of Sayreville, N.J.), C₁₂-C₁₅ alkyl benzoates (such as FINSOLV TN), caprylic/capric triglycerides, silicone oils (such as dimethicone and cyclopentasiloxane), pentaerythritol tetraoctanoate and mineral oil. Other examples of suitable oils include liquid organic ultraviolet filter commonly used for example as UV-absorbing sunscreens such as octocrylene, octyl salicylate, octyl methoxyxcinnamate, among others.

Other compounds suitable for use in the continuous oil phase include those that meet the definition of “hydrophobic compound,” but not necessarily those of “oil.” Examples of such compounds include solid sunscreens (e.g., avobenzone, oxybenzone), and any of various oil-phase rheology modifiers, particularly those oil-phase rheology modifiers suitable for increasing yield stress and/or shear modulus (G′) of the composition. Examples of components suitable for increasing yield stress and/or shear modulus include silicone elastomers, waxes, and hydrophobically-modified clays. The total concentration of oil-phase rheology modifiers may be, for example from about 3% to about 15%, such as from about 3% to about 10%, such as from about 3.5% to about 8%.

Waxes that may be suitable for increasing yield stress and/or shear modulus (G′) of the composition include waxy hydrocarbons (straight or branched chain alkanes or alkenes, ketone, diketone, primary or secondary alcohols, aldehydes, sterol esters, alkanoic acids, turpenes, monoesters), such as those having a carbon chain length ranging from C₁₂-C₃₈, silicone waxes. The wax may be a natural wax including beeswax (e.g., White Beeswax SP-422P available from Strahl and Pitsch, New York), insect waxes, sperm whale oil, lanolin, vegetable waxes such as canauba wax, jojoba oil, candelilla wax; mineral waxes such as paraffin wax; and synthetic waxes such as C30-C45 olefins and C30-C45 alkyl methicones (e.g., ST-Wax 30 available from Dow Corning of Midland, Mich.); dicaprylyl carbonate (available as CETIOL CC from Cognis Corporation of Ambler, Pa.); cetyl palmitate, lauryl palmitate, cetostearyl stearate, and polyethylene wax (e.g., PERFORMALENE 400, having a molecular weight of 400 and a melting point of 83-88° C., available from New Phase Technologies of Sugar Land, Tex.). Other suitable waxes include silicone waxes such as alkyl siloxane waxes (e.g., DC ST-30 Wax from Dow Corning of Midland Mich.), as well as C30-45 alkyl methicone and C30-45 olefin (e.g., Dow Corning AMS-C30, having a melting point of 70° C-80° C., available from Dow Corning of Midland, Mich.). The concentration of waxes may be, for example from about 0.5% to about 5.

Silicone elastomers suitable for increasing yield stress and/or shear modulus include silicone elastomers (i.e., crosslinked polyorganosiloxane elastomers) suitable for increasing yield stress and/or shear modulus include chemically or physically crosslinked molecules having at least one siloxane repeat unit, wherein the material is generally flexible and deformable and having a modulus of elasticity such that the material is resistant to deformation and has a limited ability to expand and to contract. The material is capable of returning to its original shape after it has been stretched. This elastomer is formed of polymeric chains of high molecular weight, the mobility of which is limited by a uniform network of crosslinking points. These organopolysiloxanes can be provided in the form of a powder, the particles constituting this powder having a size ranging from 0.1 to 500 μm and better still from 3 to 200 μm and beingable to be spherical, flat or amorphous with preferably a spherical shape. They can also be provided in the form of a gel comprising the elastomeric organopolysiloxane dispersed in an oilyphase. This oily phase, also known as liquid fatty phase, can comprise any non-aqueous substance or mixture of non-aqueous substances which is liquid at room temperature (25° C.).

Preferred silicone elastomers include crosspolymers of dimethicone and vinyl dimethicone (e.g., KSG 1610 and USG 107A, both from Shin-Etsu of Japan). Other silicone elastomers suitable for increasing yield stress and/or shear modulus are crosslinked elastomeric solid organopolysiloxanes that are described below with reference to W/O emulsifiers. The concentration of silicone elastomer may be from about 0.25% to about 5%, such as from about 2.75% to about 10%, such as from about 3% to about 7%, such as from about 3% to about 6% by weight of active silicone elastomer. Other silicone elastomers suitable for increasing yield stress and/or shear modulus are crosslinked elastomeric solid organopolysiloxanes that are described below as W/O emulsifiers.

Suitable hydrophobically modified clays for increasing yield stress and/or shear modulus include hydrophobically modified bentonite, such as TIXOGEL 1538 and TIXOGEL 1478, a mixture of isohexadecane, quat-90 bentonite, and propylene carbonate, available from Southern Clay Products of Gonzalez, Tex. The concentration of hydrophobically modified clay may be from about 2% to about 15%, such as from about 2% to about 10%

The emulsion is desirably stabilized by a suitable stabilizer, e.g., a water-in-oil (W/O) emulsifier. Any suitable water-in-oil emulsifier may be used in the present invention. Typical oil-in-water emulsifiers are capable of being combined with deionized water and either dimethicone or mineral oil, for example such that the relative concentration of the compound (on an active basis) is 1% by weight, the relative concentration of deionized water is about 96% by weight and the relative concentration of dimethicone or mineral oil is 3% by weight. This mixture can be agitated such that the mixture forms an emulsion of water in dimethicone or mineral oil that remains stable (no visible phase sepearation) when held at a temperature of about 25° C. for at least 24 hours. In one embodiment, the HLB (hydrophile-lipophile balance) of the emulsifier is low. For example, the emulsifier may have an HLB that is less than about 14, preferably from about 2 to about 13, more preferably from about 2 to about 10, most preferably from about 2 to about 9.

The concentration of W/O emulsifier may be varied. In certain embodiments, the W/O emulsifier is present in a concentration (on an actives basis) from about 2% to about 8%, such as from about 2% to about 8%, such as from about 4.75% to about 8%.

Suitable W/O emulsifiers include esters of glycerol and long carbon chain (fatty) acids, as well as other commonly used hydrocarbon and non-silicone W/O emulsifiers commonly used in personal care compositions. Also suitable are silicone emulsifiers such as (1) a non-crosslinked dimethicone copolyol such as alkoxy dimethicone copolyol, (2) crosslinked elastomeric solid organopolysiloxanes comprising at least one oxyalkylenated group, and combinations thereof.

Suitable non-crosslinked dimethicone copolyols that can serve as W/O emulsfiers include, for example, a mixture of dimethicone copolyol, pentacyclomethicone (D5) and water (ratio by weight 10/88/2), sold under the name DC 5225C; and a mixture of PEG-12 dimethicone copolyol sold under the name DC9011.

Particularly suitable non-crosslinked dimethicone copolyols include various silicones having pendant hydrophilic moieties that are available from Shin-Etsu Silicones of Akron, Ohio, such as linear silicones having pendant polyether groups such as KF-6028; branched polyether and alkyl modified silicones such as KF-6038; and branched polyglycerin and alkyl modified silicones such as KF-6105. Other suitable dimethicone copolyols include for example, cetyl dimethicone copolyol, such as that sold under the name Abil EM-90, bis-PEG/PPG-14/dimethicone copolyol sold under the name Abil EM-97 or such as the polyglyceryl-4 isostearate/cetyl dimethicone copolyol/hexyl laurate mixture sold under the name Abil WE 09. Abil EM-90, Abil EM-97 and Abil WE 09 are available from Evonik Goldschmidt GmbH of Essen, Germany.

In certain embodiments, the inventive compositions include a crosslinked elastomeric solid organopolysiloxanes comprising at least one oxyalkylenated group. By “crosslinked elastomeric solid organopolysiloxanes comprising at least one oxyalkylenated group,” it is meant chemically or physically crosslinked molecules having at least one siloxane repeat unit, wherein the material is generally flexible and deformable and having a modulus of elasticity such that the material is resistant to deformation and has a limited ability to expand and to contract. The material is capable of returning to its original shape after it has been stretched. This elastomer is formed of polymeric chains of high molecular weight, the mobility of which is limited by a uniform network of crosslinking points. The crosslinked elastomeric solid organopolysiloxanes useful in the composition of the invention comprise one or more oxyalkylenated groups and preferably oxyethylenated (OE) groups, for example from 1 to 40 oxyalkylene units and better still 1 to 20 oxyalkylene units, which can form polyoxyalkylene chains and in particular polyoxyethylene chains. These groups can be pendant, at the chain end or intended to bond two parts of the silicone structure. The silicon atoms carrying these groups preferably number from approximately 1 to 10. These organopolysiloxanes can be provided in the form of a powder, the particles constituting this powder having a size ranging from 0.1 to 500 μm and better still from 3 to 200 μm and being able to be spherical, flat or amorphous with preferably a spherical shape. They can also be provided in the form of a gel comprising the elastomeric organopolysiloxane dispersed in a liquid fatty phase, can comprise any non-aqueous substance or mixture of non-aqueous substances which is liquid at room temperature (25° C.). The organopolysiloxanes of the invention may be obtained according to the procedure of Examples 3, 4 and 8 of U.S. Pat. No. 5,412, 004 and of the examples of U.S. Pat. No. 5,811,487, both incorporated herein in their entirety.

Suitable elastomeric organopolysiloxanes which can be used in the composition of the invention include for example, polyether-modified crosslinked siloxanes such as KSG-210 (available as about 25% of active dimethicone crosspolymer PEG-10/15), or polyglycerin-modifed crosslinked siloxanes such as KSG-710 (available as about 25% of active polyglycerin-modified crosspolymer), both available from Shin-Etsu Silicones of Japan.

Crosslinked elastomeric solid organopolysiloxanes comprising at least one oxyalkylenated group are, in certain embodiments, particularly useful to form compositions of the present invention, in that these materials can serve both as a W/O emulsifier as well as to aid in increasing yield stress. In certain embodiments, the crosslinked elastomeric solid organopolysiloxanes comprising at least one oxyalkylenated group is present in a concentration from about 0.25% to about 5%, such as from about 1% to about 5%, such as from about 3% to about 5% by weight of active crosslinked elastomeric solid organopolysiloxanes comprising at least one oxyalkylenated group.

The total concentration of the W/O emulsifier in the composition of the invention is preferably from about 0.1% to about 10%, preferably from about 0.3% to about 5%, more preferably from about 0.4% to about 2.5% by weight.

The proportion of oil phase present in the composition may be varied, but is generally suitable to provide sufficient separation of the water phase particles, as well as spreadability and pleasant skin-feel. In certain embodiments of the invention, the amount of oil phase in the composition is from 20% to about 98%, preferably from about 30% to about 96%, more preferably from about 30% to about 80%, and most preferably from about 30% to about 55% by weight of the composition.

The water phase comprises the internal, discontinuous phase of the emulsion and comprises water and other optional ingredients that are generally hydrophilic and intimately mixed therewith. The discontinuous water phase is stabilized within the continuous oil phase as discrete regions, the majority of which preferably have a size of about 0.2 microns to about 10 microns, more preferably from about 0.5 microns to about 5 microns, most preferably from about 0.75 microns to about 5 microns.

Ingredients suitable for use in the water phase include, for example water, dissolved salts such as sodium chloride, water soluble surfactants, water-soluble preservatives and dyes, chelating agents (e g , amino acids such as glycine, edta, citrate, and the like), pH adjusters and buffers (e.g., citric acid, sodium hydroxide, bicarabonate and the like), water-soluble biologically active compounds, glycerin, glycols, and the like. In certain embodiments of the invention, the amount of water phase in the composition is from 30% to about 80%, preferably from about 40% to about 65%, more preferably from about 45% to about 60%, and most preferably from about 45% to about 55% by weight of the composition.

According to certain preferred embodiments of the invention, compositions of the present invention are “single” water-in-oil emulsions (i.e., the phase composition of the emulsion is a single water phase in a single oil phase). According to certain other embodiments of the invention, compositions of the present invention are multiple emulsions such as W/O/W emulsions or O/W/O emulsions.

In one embodiment, the compositions of the present invention may also include suspended or dispersed hydrophilic interfacial particulates. The hydrophilic interfacial particulates are generally solid at room temperature, insoluble in either the water phase or the oil phase, and have hydrophilic surfaces (e.g., have anionic, cationic, zwitterionic, or nonionic surface groups such as silanol, sulfate, sulfonate, carboxylate, phosphate, phosphonate, ammonium, including mono-, di-, and trialkylammonium species, pyridinium, imidazolinium, amidinium, poly(ethyleneiminium), ammonioalkylsulfonate, ammonioalkylcarboxylate, amphoacetate, or poly(ethyleneoxy)sulfonyl moieties) that tend to mix intimately with water. The majority of the hydrophilic interfacial particulates may have a particle size that is from about 1 micron to about 50 microns, such as from about 2 to about 20 microns. In one embodiment, the hydrophilic interfacial particulates have a surface area (as measured by BET) that is at least about 1 m²/g, such as at least about 5 m²/g. In one embodiment the density (i.e., particle density, not bulk density) of the hydrophilic interfacial particulates is less than about 5 g/cc, such as less than about 3 g/cc, such as from about 1 g/cc to about 4 g/cc, such as from about 1.5 g/cc to about 3 g/cc. The total amount of the hydrophilic interfacial particulates in the composition of the invention is preferably from about 0.2% to about 5%, preferably from about 0.5% to about 3%, more preferably from about 1% to about 3% by weight of the composition.

In one embodiment, the hydrophilic interfacial particulates comprise a non-oxide pigment such as a silica or aluminosilicate particulate, such as an uncoated (spherical) silica, e.g., MSS-500W available from Kobo of South Plainfield, New Jersey, or a fumed silica such as Aerosil A200 from Degussa.

### Rheology

The inventors have found that, according to certain embodiments of the invention, in order to form emulsions including water-susceptible particulates that are chemically and phase stable, the emulsion should have a yield stress that is at least about 20 Pascals. According to certain embodiments of the invention, the yield stress is from about 20 Pa to about 200 Pa, such as from about 20 Pa to about 100 Pa.

A suitable method for determining yield stress and other rheological paraemeters employs a parallel plate rheometer such as Rheometrics RFS II (Rheometrics Scientific, Piscataway, N.J.). The rheometer and samples are equilibrated at 25 C and all tools are cleaned. The plate diameter is set at 25.0 mm. The sample is gently mixed in the sample container. Using a clean spatula, sample is withdrawn and loaded onto lower plate and upper tool is set to 1.0 mm spacing. Samples edges are cleaned with a wipe and upper tool is brought to final spacing of 0.8 mm. A vapor hood is installed and test is begun. After completion of test, sample is removed and tools are cleaned. Shear stress/shear rate profiling is performed using an accelerated/decelerated flow test (thixotropic loop; 0 to 100 to 0 s⁻¹ with 100 second acceleration/deceleration times and no delay) is selected covering the shear rate range of 100 s⁻¹ in a 100 second time interval. Yield stress is estimated from the accelerated shear rate ramp at the inception of flow. This can be readily estimated from viewing the shear stress versus shear rate plot using a logarithmic scale.

For test samples that are too “thin” in consistency to get consistent reading with the 15 mm parallel plate geometery, as an alternative a couette geometry (34 mm cup, 32 mm bob, 33.4 mm length) may be employed.

Shear storage modulus, G′ is a measure of the emulsion's elastic modulus frequency response. The inventors have found that according to certain embodiments of the invention, the emulsion should have a shear storage modulus that is at least about 80 Pascals. According to certain embodiments of the invention, the shear storage modulus, G′ is from about 80 Pa to about 1000 Pa, such as from about 80 Pa to about 650 Pa.

Tan delta, is another measure of the emulsion's elastic modulus frequency response. In certain embodiments, tan delta of the emulsion is from about 0.05 to about 0.4, such as from about 0.1 to about 0.35. Tan delta may be determined using an identical method to the one described above for yield stress, except that both G′ and G″ are read off directly from the plot at a frequency of 0.1 radians per second (or alternatively at 1 radian per second, if no reading can be ascertained at 0.1 radian per second). Tan delta is the ratio of loss modulus to storage modulus, G″/G′.

G′ and G″ may be determined using an similar method to the one described above for yield stress, for example by doing a frequency sweep starting at 0.1 radians per second, with a 60 second delay and strain set at 0.005. G′ at a frequency of 0.1 radians per second is reported. Tan delta, another measure of the emulsion's elastic modulus frequency response, is the ratio of loss modulus to storage modulus, G″/G′. To compute tan delta G″ and G′ are determined at 0.1 radians per second and the quotient is calculated. If no reading can be ascertained for G′ or G″ at 0.1 radian per second, then these parameters are measured at 1 radian per second.

### Other Ingredients

In one embodiment, the composition comprises an additional active agent. As used herein, “additional active agent” means a compound (e.g., synthetic or natural) that provides a cosmetic or therapeutic effect on the skin, such as a therapeutic drug or cosmetic agent. Examples of therapeutic drugs include small molecules, peptides, proteins, nucleic acid materials, and nutrients such as minerals and extracts. Other examples of additional active agents include anti-aging agents, anti-inflammatory agents, anti-acne agents, antimicrobial agents, antioxidants, external analgesics, vitamins and skin lightening agents.

Examples of suitable anti-aging agents include, but are not limited to: inorganic sunscreens such as titanium dioxide and zinc oxide; organic sunscreens; retinoids; alpha hydroxy acids and their precursors such as glycolic acid, pyruvic acid, beta hydroxy acids such as beta-hydroxybutyric acid; tetrahydroxypropyl ethylene-diamine, N,N,N′,N′-Tetrakis(2-hydroxypropyl)ethylenediamine (THPED); and botanical extracts such as green tea, soy, milk thistle, algae, aloe, angelica, bitter orange, coffee, goldthread, grapefruit, hoellen, honeysuckle, Job's tears, lithospermum, mulberry, peony, puerarua, nice, and safflower; and salts, derivatives and prodrugs thereof. Examples of anti-inflammatory agents, include, but are not limited to, suitable steroidal anti-inflammatory agents such as corticosteroids such as hydrocortisone. Examples of vitamins include Vitamin E, Vitamin A, Vitamin C, Vitamin B, and salts or derivatives thereof such as ascorbic acid di-glucoside and vitamin E acetate or palmitate.

The amount of the additional active agent in the composition will depend on the active agent, other ingredients present in the composition, and the desired benefits of the composition. In one embodiment, the composition contains a safe and effective amount of the additional active agent, for example, from about 0.001 percent to about 20 percent, by weight, such as from about 0.01 percent to about 10 percent, by weight, of the composition.

In one embodiment, the emulsion includes a plant extract or other natural ingredient. Examples of plant extracts include, but are not limited to, soy, glycine soja, oatmeal, and aloe vera.

In another embodiment, the emulsion includes a feverfew extract. As used herein, “feverfew extract” is a blend of compounds isolated from a plant from the Chrysanthemum or Tanacetum genus (hereinafter referred to as feverfew). Examples of feverfew include, but are not limited to, Chrysanthemum parthenium, Tanacetum parthenium, or Matricania parthenium, as well as those listed in CRC Ethnobotany Desk Reference 1998, ed. Timothy Johnson, p 198-199, 823-824, 516-517 (CRC Press, Boca Raton, Fla., USA 1998) and the The Plant Names Project (1999), International Plant Names Index, published on the Internet; http://www.ipni.org [accessed Jan. 11, 2001]. The feverfew extract may be substantially free of parthenolide. What is meant by “substantially free of parthenolide” is that the composition comprises, by weight, less than 0.1%, preferably below 0.01%, more preferably below 0.001% or does not comprise any parthenolide. In one embodiment, the composition does not comprise parthenolide.

Other optional ingredients include abrasives, absorbents, aesthetic components such as chelating agents, skin sensates, astringents, anti-caking agents, antifoaming agents, binders, buffering agents, bulking agents, chemical additives, cosmetic biocides/preservatives, colorants, additional emulsifiers, film formers or materials, e.g., polymers, for aiding the film-forming properties and substantivity of the composition, opacifying agents, propellants, skin-conditioning agents (e.g., humectants, including miscellaneous and occlusive), skin soothing and/or healing agents, and skin treating agents.

The emulsion may include one or more pigments (e.g., non-metallic) such as inorganic pigments, lake pigments, and interference pigments. Inorganic pigments include titanium dioxide and mica as well as color pigments such as iron oxides, including red and yellow iron oxides, ultramarine and chromium or chromium hydroxide colors, and mixtures thereof The emulsion may also include a lake pigment. Examples of lake pigments include organic dyes such as azo, indigoid, triphenylmethane, anthraquinone, and xanthine dyes that are designated as D&C and FD&C blues, browns, greens, oranges, reds, yellows, etc., precipitated onto inert binders such as insoluble salts. In one embodiment, the lake pigment is selected from Red 6, Red 7, Yellow 5 and Blue #1. Examples of interference pigments include those containing mica substrates, bismuth oxycloride substrates, and silica substrates, for instance mica/bismuth oxychloride/iron oxide pigments commercially available as CHROMALITE pigments (BASF), titanium dioxide and/or iron oxides coated onto mica such as commercially available FLAMENCO pigments (BASF), mica/titanium dioxide/iron oxide pigments including commercially available KTZ pigments (Kobo products), CELLINI pearl pigments (BASF), and borosilicate-containing pigments such as REFLECKS pigments (BASF). The total concentration of pigment may range from about 0.05% to about 15% weight percent inorganic pigments, such as from about 2% to about 12%.

### Product Forms and Uses

Compositions of the present invention may take any one of a wide variety of forms that include but are not limited to forms generally suitable for “leave-on” products such as lotions, creams, gel-creams, sticks, sprays, pastes, mousses and moisturizers. In another embodiment the product form may be suitable for a rinse-off product such as washes, shampoos, and other cleansing liquids. Other suitable forms include impregnated wipes, patches, hydrogels or wound dressings; and adhesives.

Also suitable are color cosmetics. As used herein, “color cosmetic” means a composition for application to the hair, nails and/or skin, especially the face, which contains at least about 0.01% and up to about 50% of pigment (such as 0.5% to about 50%, such as from about 1% to about 30%), especially color pigments. Color cosmetics include, but are not limited to, foundations, concealers, primers, blush, mascara, eyeshadow, eyeliner, lipstick, nail polish and tinted moisturizers. The present invention is particularly suited for use with foundations, concealers, and primers.

As used herein, “foundation” means a liquid, solid, or semi-solid cosmetic composition for imparting color to the skin, especially the face. It may be in the form of, for example, a lotion, cream, stick, or paste.

As used herein, “concealer” means a liquid, paste, or semi-solid cosmetic composition for imparting color to the skin, containing a relatively high level of pigments having opacity, such as titanium dioxide, typically used prior to applying foundation, for example for concealing age or acne spots or scars.

As used herein, “primer” means a liquid, paste, or semi-solid cosmetic composition for application directly to the skin underneath foundations and/or concealers. Primers ease the application of foundation (or other skin care composition) onto the skin, even out skin tone, and increase the longevity of skin care compositions applied over the primer. Primers also may be used to smooth fine lines, such as around the mouth. A lip primer used underneath lipstick can maintain lip color and prevent feathering of the lipstick. Foundation primer used around the eye area can decrease creasing of eyeshadow. Use of a foundation primer may also decrease the amount of foundation required to achieve the same effect. Primers typically comprise waxes, polymers, and silicones.

### Process of Making

The inventors have found that surprisingly stable emulsions that include water-susceptible particulates can be formed by first forming a water-in-oil emulsion. The water-in-oil emulsion can be formed using conventional techniques known in the art of cosmetic formulation. For example, this may include combining one or more hydrophobic compounds to form an oil phase. In one embodiment, a W/O emulsifier is added to the oil phase. In another embodiment, one or more (e.g. pre-ground) pigments such as inorganic, lake, and/or interference pigments are added to the oil phase. Separately, water and optional hydrophilic ingredients are combined to form a water phase.

The water phase and the oil phase may be separately heated to a substantially common temperature, e.g., greater than about 50° C., such as about 85° C. The water phase may then be added to the oil phase and allowed to mix for a period of time sufficient to form a W/O emulsion.

According to certain embodiments of the invention, after the W/O emulsion is formed, the water susceptible particulate is added to the W/O emulsion. After forming the W/O emulsion, but prior to adding the water susceptible particulate, the W/O emulsion may be allowed to cool such as to below 30° C. Furthermore, during addition of the water susceptible particulate, mixing may be maintained for example using a stirrer at a speed of rotation that is less than about 100 rpm, such as about 50 rpm. The water susceptible particulate may added all at once or gradually over a period of 15 to 60 minutes. In another embodiment, one or more (pre-ground) pigments are added after the emulsion is formed.

The step of homogenization, an intensive blending typically applied to an emulsion after the emulsion has been formed, is used to reduce emulsion particle size. In certain embodiments of the invention, the step of homogenization is omitted.

Compositions of the present invention are surprisingly stable. For example, for embodiments in which the water-susceptible particulate is a galvanic particulate, the compositions have one or more of: greatly reduced or eliminated outgassing, reduced or eliminated color changes, and increased topical anti-inflammatory activity (all of which serve as an indicators of the stability of the zinc-copper powder, i.e., the zinc-copper galvanic particulate).

The following non-limiting examples further illustrate the invention.

### EXAMPLES

### Example I: Inventive Examples

The following compositions, Inventive Example Ex. 1-2, shown in Table 1, were made according to the invention. They contained zinc-copper powder, a water-susceptible particulate.

TABLE 1

| Trade Name | CTFA Name | Ex. 1 | Ex. 2 |
|---|---|---|---|
| KSG-210 | Dimethicone/PEG-10/15 Crosspolymer | 22.0 | 15.0 |
| | (25%); Dimethicone ; | | |
| KF-6028 | PEG-9 Polydimethylsiloxyethyl | 1.0 | 1.0 |
| | dimethicone | | |
| ABIL WE 09 | Polyglyceryl-4 Isostearate (40%); Cetyl | 1.5 | 1.5 |
| | PEG/PPG-10/1 Dimethicone (30%); | | |
| | Hexyl Laurate (30%) | | |
| DC 2-1184 | Dimethicone (40%); Trisolxane (60%) | 14.7 | 4.0 |
| Dow Corning | C30-45 Alkyl Methicone; | 0.7 | 4.0 |
| AMS-C30 | C30-45 Olefin | | |
| Cosmetic | | | |
| Dow Corning 200 | Dimethicone | | 4.0 |
| Fluid | | | |
| Trivent PE 48 | Pentaerythrityl tetrahexanoate | | 5.0 |
| TMF-1.5 | Methyl trimethicone | 10.0 | 4.0 |
| Cetiol CC | Dicaprylyl carbonate | | 2.0 |
| Aerosil 200 | Silica | 0.3 | |
| MSS-500W | Silica | | 1.0 |
| Nipasol M | Propyl paraben | 0.2 | |
| EUXYL PE 910 | Phenoxyethanol; ethylhexylglycerin | | 0.8 |
| Elestab CPN Ultra | Chlorphenesin | | 0.25 |
| Pure | | | |
| Water | Water | 43.0 | 49.85 |
| Sodium chloride | Sodium chloride | 0.5 | 0.5 |
| USP | | | |
| Butylene glycol | Butylene glycol | 3.0 | 3.0 |
| Glycerin | | | 2.0 |
| Versene NA | Disodium EDTA | 0.1 | 0.1 |
| Zinc-Copper | Zinc; Copper | 3.0 | 2.0 |
| Powder | | | |
| TOTAL | | 100.0 | 100.0 |

Inventive Example, Ex. 1 was prepared by forming a water phase by combining water, EDTA, sodium chloride, butylene glycol, silica to a vessel and heating to 80° C. An oil phase was prepared by combining KSG-210, KF-6028, Abil WE09, DC 2-1184, AMS-CS 30, TMF 1.5 and propylparaben to a vessel and heating to 80° C. under propeller mixing. When both phases reached 85° C., the water phase was slowly added to the oil phase. After emulsification, the heat was shut off and the emulsion was allowed to mix for 10 minutes. The emulsion was then homogenized using a Silverson homogenizer for 5 minutes. The homogenized emulsion was then again agitated with a propeller mixer and allowed to cool to 25° C. The zinc/copper powder was added under and mixed until uniform.

Inventive Example, Ex. 2 was prepared by forming a water phase by combining water, EDTA, sodium chloride, phenoxyethanol, butylene glycol, glycerine, MSS-500W and Elestab CPN ultra pure to a vessel and heating to 85° C. An oil phase was prepared by combining KSG-210, KF-6028, Abil WE09, DC 2-1184, dimethicone, Trivent PE48, AMS-CS 30, TMF 1.5 & Cetiol CC to a vessel and heating to 85° C. under propeller mixing. When both phases reached 85° C., the water phase was slowly added to the oil phase. After emulsification, the heat was shut off and the emulsion was allowed to mix for 10 minutes. The mixing was adjusted to a slow sweep at 50 rpm and the mixture was allowed to cool to 28° C. The zinc/copper powder was added under slow sweep mixing, and the composition was allowed to mix until uniform.

### Example II: Comparative Example

The following composition, Comparative Example, Comp. 1, shown in Table 2 was prepared. It also contained zinc-copper powder.

TABLE 2
Comparative Example, Comp. 1

| Trade Name | Chemical/INCI Name | Comp. 1 |
|---|---|---|
| Atlas White AS | Titanium dioxide | 10 |
| Unipure yellow | Iron Oxides ( Yellow 42) & Triethoxy | 2 |
| LC 182 AS | Caprylylsilane | |
| Unipure red LC | Iron Oxides (Red 101) Triethoxy | 0.6 |
| 381 AS | Caprylylsilane | |
| Unipure black LC | Iron Oxides (Cl 77499) & Triethoxy | 0.2 |
| 989 AS | Caprylylsilane | |
| DM-FLUID A-6cs | Dimethicone | 10 |
| USG 107A | Dimethicone/Vinyl Dimethicone | 20 |
| | Crosspolymer | |
| Permethyl 101A | Isohexadecane | 5 |
| KF 8020 | Amino dimethicone | 5 |
| Permethyl 99A | Isododecane | 5 |
| KF 6038 | Lauryl PEG-9 Polydimethylsiloxyethyl | 4.5 |
| | Dimethicone | |
| Gransurf 67 | PEG-10 Dimethicone | 2.5 |
| Zinc-Copper | Zinc; Copper | 1 |
| Powder | | |
| DI Water | Water | 33.6 |
| NaCl | Purified | 0.5 |
| | Sodium | |
| | Chloride USP | |
| Versene NA | Disodium EDTA | 0.1 |
| TOTAL | | 100.0 |

Comparative Example, Comp. 1 was prepared by grinding phase A (titanium dioxide through and including dimethicone) through a roller mill. Phase A was added to phase B (Dimethicone/Vinyl Dimethicone Crosspolymer through zinc/copper powder) and heated to 60° C. Phase C (water through EDTA) was mixed and heated to 60° C. Phase C was added to phase A/B mixture, and allowed to mix for an additional 15 minutes after adding phase C. The mixture was then homogenized for 3 mins at 30° C.

### Example III: Inventive Examples

Inventive Example Ex. 3-4 shown in Tables 3-4 were prepared, according to embodiments of the invention described herein. They also contained zinc-copper powder.

TABLE 3
Inventive Example, Ex. 3

| Trade Name | Chemical/INCI Name | Ex. 3 |
|---|---|---|
| Unipure White | Titanium dioxide | 7.59 |
| 987 AS | | |
| Unipure yellow | Iron Oxides ( Yellow 42) & Triethoxy | 0.48 |
| LC 182 AS | Caprylylsilane | |
| Unipure red LC | Iron Oxides (Red 101) Triethoxy | 0.15 |
| 381 AS | Caprylylsilane | |
| Unipure black LC | Iron Oxides (Cl 77499) & Triethoxy | 0.07 |
| 989 AS | Caprylylsilane | |
| DC 200 5 cst Fluid | Dimethicone (5 cst fluid) | 8 |
| DC 200 5 cst Fluid | Dimethicone (5 cst fluid) | 7.91 |
| Tieovil FIN | C12-15 Alkyl Benzoate, Titanium | 8 |
| | Dioxide, Polyhydroxystearic Acid, | |
| | Aluminum Stearate, Alumina | |
| KF 6028 | PEG 9 dimethicone | 1 |
| KSG 1610 | Dimethicone/Vinyl Dimethicone | 2 |
| | Crosspolymer; methyl triemthicone | |
| KSG 210 | Dimethicone cosspolymer PEG-10/15 | 9 |
| USG 107A | Dimethicone/Vinyl Dimethicone | 6 |
| | Crosspolymer | |
| KF 6038 | Lauryl PEG-9 Polydimethylsiloxyethyl | 1.3 |
| | Dimethicone | |
| Abil WE-09 | Polyglyceryl-4 Isostearate; Cetyl | 1.2 |
| | PEG/PPG-10/1 | |
| | Dimethicone; Hexyl Laurate | |
| AMS C30 | alkyl silicone wax | 0.6 |
| A 200 | Silica | 0.3 |
| Tinogard TT | Pentaerythrityl Tetra-di-t-butyl | 0.1 |
| | Hydroxyhydrocinnamate | |
| Propyl paraben | Propyl paraben | 0.2 |
| DI Water | Water | 44 |
| NaCl | Purified | |
| | Sodium | |
| | Chloride USP | |
| Versene NA | Disodium EDTA | 0.1 |
| Zinc-Copper | Zinc; Copper | 1 |
| Powder | | |
| TOTAL | | 100.0 |

Inventive Example, Ex. 3 was prepared by mixing together an oil phase (second dimethicone through and including paraben) in the main kettle and heating to 80 C. A water phase was prepared by mixing water through EDTA and heating to 80 C. Over a 20 minute period the water phase was added to the oil phase and the temperature was maintained above 70 C. The mixture was then mixed for an additional 15 minutes at 75 C. The heat was removed and a second oil phase having dispersed pigment (titanium dioxide through and including the first dimethicone) was added at a temperature of 60-70 C. This was allowed to mix for 20 minutes and then homogenized for 3 minutes at 60 C. The batch was allowed to cool to 25 C and zinc/copper powder was added, mixed slowly at 70-100 rpm using a paddle sweep.

TABLE 4
Inventive Example, Ex. 4

| Trade Name | Chemical/INCI Name | Ex. 4 |
|---|---|---|
| Unipure White | Titanium dioxide | 6.563 |
| 987 AS | | |
| Unipure yellow | Iron Oxides ( Yellow 42) & Triethoxy | 0.953 |
| LC 182 AS | Caprylylsilane | |
| Unipure red LC | Iron Oxides (Red 101) Triethoxy | 0.37 |
| 381 AS | Caprylylsilane | |
| Unipure black LC | Iron Oxides (Cl 77499) & Triethoxy | 0.114 |
| 989 AS | Caprylylsilane | |
| DC 200 | Dimethicone (5 cst fluid) | 2.2 |
| DC 556 Cosmetic | Phenyl Trimethicone | 1 |
| Grade Fluid | | |
| TMF 1.5 | Methyl Trimethicone | 3.5 |
| Trivent PE 48 | pentaerythrityl tetranoctanoate | 2 |
| Elefac I-205 | Octyldodecyl neopentanoate | 3 |
| KP 545L | Silicone Acrylate | 5 |
| Tieovil 50 FIN | C12-15 Alkyl Benzoate, Titanium | 7 |
| | Dioxide, Polyhydroxystearic Acid, | |
| | Aluminum Stearate, Alumina | |
| KSG 1610 | Dimethicone/Vinyl Dimethicone | 5 |
| | Crosspolymer; methyl triemthicone | |
| KSG 210 | Dimethicone cosspolymer PEG-10/15 | 4.9 |
| USG 107A | Dimethicone/Vinyl Dimethicone | 4.9 |
| | Crosspolymer | |
| KF 6038 | Lauryl PEG-9 Polydimethylsiloxyethyl | 1.5 |
| | Dimethicone | |
| Abil WE-09 | Polyglyceryl-4 Isostearate; Cetyl | 1.2 |
| | PEG/PPG-10/1 | |
| | Dimethicone; Hexyl Laurate | |
| AMS C30 | alkyl silicone wax | 1 |
| A 200 | Silica | 0.3 |
| Tinogard TT | Pentaerythrityl Tetra-di-t-butyl | 0.1 |
| | Hydroxyhydrocinnamate | |
| DI Water | Water | 42 |
| Butylene Glycol | Butylene Glycol | 1 |
| | Glycerine | 1.5 |
| NaCl | Purified | 1 |
| | Sodium | |
| | Chloride USP | |
| Versene NA | Disodium EDTA | 0.1 |
| Optiphen Plus | Phenoxyethanol (and) Caprylyl Glycol | 1 |
| | (and) Sorbic Acid | |
| Orgasol EXD | Nylon | 0.3 |
| 2002 D | | |
| Asensa DS 912 | Zeolite | 0.45 |
| Sericite PHN | Mica | 1 |
| Zinc-Copper | Zinc; Copper | 1 |
| Powder | | |
| TOTAL | | 100.0 |

Inventive Example, Ex. 4 was prepared by mixing together an oil phase (methyl trimethicone through and including TINOGARD) in a main kettle and heating to 80 C. A water phase was prepared by mixing water through OPTIPHEN and heating to 80 C. Over a 20 minute period the water phase was added to the oil phase and the temperature was maintained above 70 C.

The mixture was then mixed for an additional 15 minutes at 75 C. The heat was removed and a second oil phase having dispersed pigment (titanium dioxide through and including the phenyl trimethicone) was added at a temperature of 60-70 C. This was allowed to mix for 20 minutes and then homogenized for 3 minutes at 60 C. A powder phase (nylon including mica) was added and mixed for 10 minutes. The batch was allowed to cool to 40 C and was then homogenized for 10 minutes. The batch was allowed to cool to 25 C and zinc/copper powder was added, mixed slowly at 50 rpm using a paddle sweep.

### Example V: Evaluation of Inventive and Comparative Examples

Inventive Example, Ex. 2, Inventive Example, Ex. 3 and Comparative Example, Comp. 1 were visually evaluated for stability and were also tested for topical anti- inflammatory activity on human epidermal equivalents (using the test method described in published PCT patent application, WO2009/045720, Example 11 “Anti-Inflammatory Activity on Release of UV-Induced Pro-inflammatory Mediators on Reconstituted Epidermis”). Topical anti-inflammatory activity included comparing example formulations to placebos (identical to test examples, but with no galvanic particulate).

Specifically, in anti-inflammatory activity was evaluated on human epidermal equivalents. Epidermal equivalents (EPI 200 HCF), multilayer and differentiated epidermis consisting of normal human epidermal keratinocytes, were purchased from MatTek (Ashland, Mass.). Upon receipt, epidermal equivalents were incubated for 24 hours at 37° C. in maintenance medium without hydrocortisone. Equivalents were topically treated (2 mg/cm²) with test samples in 70% ethanol/30% propylene glycol vehicle 2 hours before exposure to solar ultraviolet light (1000W-Oriel solar simulator equipped with a 1-mm Schott WG 320 filter; UV dose applied: 70 kJ/m2 as measured at 360 nm). Equivalents were incubated for 24 hours at 37° C. with maintenance medium then supernatants were analyzed for IL-1α cytokine release using commercially available kits (Millipore Corp., Billerica, Mass.).

Inventive Example, Ex. 2 and Inventive Example, Ex. 3 were evaluated at 4 wks and 12 weeks at room temperature respectively and were found to be visually stable and showed no evidence of outgassing (bubble formation). Furthermore, Inventive Example, Ex. 2 was evaluated for I1-1 activity 3 weeks after formulation and reduced the I1-1 response to 29.1% of placebo, indicating activity of the galvanic particulate. In addition, Inventive Example, Ex. 3 evaluated for I1-1 activity 4 weeks at 50 C and room temperature reduced to 55.4% and 44.6% of the placebo respectively. In addition, Inventive Example, Ex. 3 was also evaluated for I1-1 activity 12 weeks at 40 C reduced to 21% of the placebo.

By way of contrast, Comparative Example, Comp. 1 showed evidence of outgassing as well as the formation of a white residue when evaluated both after 4 weeks and at room temperature and after 2 weeks exposure to 40 C. In addition, Comparative Example, Comp. 1 did poorly when tested after 5 weeks at room temperature and 50 C increased IL-1 activity to 12% and 25% respectively compared to the placebo.

These results suggest that adding galvanic particulate after the formation of the W/O emulsion) may stabilize the emulsion.

### Example VI: Preparation and Evaluation of Inventive and Comparative Examples

Additional Inventive Examples and Comparative Examples were prepared. Specifically, a reference formula was prepared with the ingredients shown in Table 5. A water phase was prepared by mixing ingredients from water through chlorphenesin. The water phase was then heated to 85 C. An oil phase was prepared by mixing Dimethicone & Dimethicone PEG-10/15 Crosspolymer through Dicaprylyl Carbonate. The oil phase was then heated to 85 C. When both phases had reached 85 C, the water phase was slowly added to the water phase, to achieve a uniform appearance, while stirring. The heat was then removed, and the emulsion was allowed to mix for ten minutes. The mixing rate was reduced to 50 rpm and the emulsion was allowed to cool to 28 C. While continuing to mix at 50 rpm, the zinc copper powder was added to the cooled emulsion.

TABLE 5
Inventive Example, Ex. 5

| Trade Name | Chemical Name/INCI | % |
|---|---|---|
| Water | water | 50.9 |
| Edta | disodium ethylenediamine | 0.1 |
| | tetraacetate | |
| Sodium Chloride | sodium chloride | 0.5 |
| Euxyl PE9010 | phenoxyethanol/ethylhexylglycerin | 0.8 |
| 1,3 butylene glycol | butylene glycol | 3 |
| Glycerine | glycerine | 2 |
| MSS-500W | silica | 1 |
| Elestab CPN Ultra Pure | chlorphenesin | 0.25 |
| KSG-210 | Dimethicone & Dimethicone PEG- | 15 |
| | 10/15 Crosspolymer | |
| KF-6028 | PEG-9 Polydimethylsiloxyethyl | 1 |
| | Dimethicone | |
| KF 6038 | Lauryl PEG-9 | 0 |
| | Polydimethylsiloxyethyl | |
| | Dimethicone | |
| Abil WE09 | Polyglyceryl-4 Isostearate; Cetyl | 1.5 |
| | PEG/PPG-10/1 Dimethicone; Hexyl | |
| | Laurate | |
| Xiameter PMX-1184 Fluid | Dimethicone; Trisiloxane | 4 |
| DC 5 cts | dimethicone | 4 |
| Trivent PE48 | pentaerythrityl tetranoctanoate | 5 |
| DC ST-30 WAX | Alkyl siloxane wax | 4 |
| TMF 1.5 | Methyl Trimethicone | 4 |
| CF-0074 | Dimethicone Crosspolymer | 0 |
| Cetiol CC | Dicaprylyl Carbonate | 2 |
| Zinc-Copper Powder | zinc; copper | 1 |
| TOTAL | — | 100 |

Other examples were prepared in a manner similar to Inventive Example, Ex. 5. Specifically, Inventive Example, Ex. 6 had KS2-210 and KF-6028 at 2%, KF-6038 at 3%, Xiameter PMX-1184 fluid at 7%, Trivent at 8%, methyl trimethicone at 7%, 0% alkyl siloxane wax instead of 4%, and the dimethicone was adjusted up (“q.s.”) from 4% to 8% to compensate. Comparative Example, Comp. 2 was prepared in a manner similar to Inventive Example, Ex. 5, except that the KSG-210 was reduced to 5%, and was q.s. with dimethicone (dimethicone was increased from 4% to 14%). Comparative Example, Comp. 3 was prepared in a manner similar to Inventive Example, Ex. 5, except that KSG-210 was reduced to 0%, and q.s. with dimethicone. Inventive Example, Ex. 7 had 0.5% silica, q.s. with water. Inventive Example, Ex. 8 had 0% silica, q.s. with water.

Example, Ex. 8a was identical to Inventive Example, Ex. 5, except that the zinc-copper powder was added to the oil phase of the emulsion. Inventive Example, Ex. 9 was homogenized for 10 minutes using a Homogenizer Mixer (Greerco, Model 1L, by Chemieer, Inc.) after cooling down to ambient temperature. Inventive Examples, Ex. 10-12 had their levels of sodium chloride adjusted to 0%, 2.0% and 0.1%, respectively, and q.s. with water. Inventive Examples, Ex. 13 had 0% Ximaeter, 0% DC 5 and 0%TMF 1.5; and the formula was adjusted by increasing Cetiol CC to 8% and increasing Trivent to 11%. Inventive Examples, Ex. 14-15 had their levels of zinc-copper powder increased to 3% and 5% respectively, q.s. with water. Comparative Examples, Comp. 5 had KSG-210 level of 0%, KF-6028 at 2%, KF-6038 at 3%, and CF-0074 at 11%. Comp. 6 had KSG-210 levels of 0%, KF-6028 at 2%, KF-6038 at 3%, and CF-0074 at 11%, Trivent of 8%, dimethicone of 7%, Cetiol CC of 5%, and methyl triemthicone of 7%.

A summary of the Inventive Examples Ex. 5 through Ex. 17, as well as Comparative Examples, Comp. 2 through Comp. 6 are shown in Table 6. Also included in Table 6 are results of a stability test and measurements of three rheological parameters, yield stress, shear modulus and tan delta.

The yield stress, shear modulus, and tan delta were determined using the methods described in the specification above. Stability was determined by placing samples of the various example compositions at elevated temperature, 50 C, for 4 weeks. The samples were then removed from elevated temperature and allowed to cool to ambient temperature and were then visually examined for settling of powder, outgassing (by removing the tops of the container), or phase separation. Any significant settling, outgassing, or phase separation was recorded as a “Fail.”

TABLE 6
Inventive Examples and Comparative Examples

| | | | Yield | Shear | |
|---|---|---|---|---|---|
| | | | Stress | Modulus, G′ | Tan |
| Example | Description | Stability | (Pa) | (Pa) | Delta |
| Ex. 5 | Reference | Pass | — | — | — |
| Ex. 6 | Replace wax with dimethicone | Pass | 22.1 | 88.27 | 0.14 |
| Comp. 2 | Reduce dimethicone/vinyl | Fail | Very low* | 12.72 | 0.44 |
| | dimethicone crosspolymer | | | | |
| Comp. 3 | Remove dimethicone/vinyl | Fail | Very low* | — | — |
| | dimethicone crosspolymer | | | | |
| Ex. 7 | Reduce silica | Pass | 66.1 | 418.11 | 0.12 |
| Ex. 8 | Remove silica | Pass | 95.2 | 622.16 | 0.09 |
| Ex. 8a | Add galvanic powder to oil | Pass** | 59.5 | 397.83 | 0.17 |
| | phase | | | | |
| Ex. 9 | Homogenize after forming | Pass | 88.6 | 469.69 | 0.12 |
| | emulsion | | | | |
| Ex. 10 | Additional sodium chloride | Pass | 78.2 | 442.27 | 0.13 |
| Ex. 11 | Additional sodium chloride | Pass | 84.8 | 411.12 | 0.11 |
| Ex. 12 | Additional sodium chloride | Pass | 80.1 | 405.73 | 0.11 |
| Ex. 13 | Replace silicone oils with | Pass | 81.9 | 246.85 | 0.19 |
| | hydrocarbon oils | | | | |
| Ex. 14 | Increase galvanic powder | Pass | 88.6 | 450.22 | 0.10 |
| Ex. 15 | Increase galvanic powder | Pass | 82.9 | 435.64 | 0.13 |
| Comp. 5 | Change dimethicone | Fail | Very low* | — | — |
| | crosspolymers/and | | | | |
| | crosspolymer/emulsifier | | | | |
| Comp. 6 | Remove rheology modifiers | Fail | Very low* | 1.53 | 0.65 |
| Ex. 16 | Reduce dimethicone/vinyl | Pass | 38 | 290.5 | 0.2555 |
| | dimethicone crosspolymer | | | | |
| Ex. 17 | Reduce dimethicone/vinyl | Pass | 25.95 | 186.8 | 0.3255 |
| | dimethicone crosspolymer | | | | |

Dashed lines in Table 6 indicate that the example was not tested. “Very low*” indicated that the examples was very thin, and it was not possible to measure yield stress. Comparative Examples, Comp. 2 failed due to settling and Comp. 3, Comp. 5 and Comp. 6 failed due to phase instability (i.e., no uniform emulsion could be formed). While Example Ex. 8a passed the elevated temperature stability test, additional stability testing (holding at ambient for about five weeks, followed by about 48 hours of agitation at 40 C) showed phase separation.

It can be seen from Table 6 that failures shared the characteristic that they included an insufficient amount of oil-phase rheology modifier (for example, KSG-210). As such, the yield stress of the comparative examples was below about 20 Pa.

It is understood that while the invention has been described in conjunction with the detailed description thereof, that the foregoing description is intended to illustrate and not limit the scope the invention, which is defined by the scope of the appended claims. Other aspects, advantages, and modifications are within the claims.

We claim:

1. A method of making a water-in-oil emulsion composition, comprising:
forming a water-in-oil emulsion; and
after forming the water-in-oil emulsion, adding a galvanic particulate to the water-in-oil emulsion.

2. The method of claim 1, wherein the water-in oil emulsion has a yield stress of at least about 20 Pa.

3. The method of claim 1, wherein the water-in oil emulsion has a yield stress from about from about 20 Pa to about 200 Pa.

4. The method of claim 1, wherein the water-in oil emulsion has a yield stress from about from about 20 Pa to about 100 Pa.

5. The method of claim 1, wherein the water-in-oil emulsion comprises an oil-phase rheology modifier selected from a group consisting of a silicone elastomer, a wax, a hydrophobically-modified clay, and combinations thereof.

6. The method of claim 1, wherein the water-in-oil emulsion comprises from about 3% to about 15% by weight of an oil-phase rheology modifier selected from a group consisting of a silicone elastomer, a wax, a hydrophobically-modified clay, and combinations thereof.

7. The method of claim 1, wherein the water-in-oil emulsion comprises from about 3% to about 10% of an oil-phase rheology modifier selected from a group consisting of a silicone elastomer, a wax, a hydrophobically-modified clay, and combinations thereof.

8. The method of claim 1, wherein the galvanic particulate is a zinc-copper particulate.

9. The method of claim 1, wherein the water-in-oil emulsion comprises a silicone elastomer.

10. The method of claim 1, wherein the water-in-oil emulsion comprises from about 0.25% to about 5% of a silicone elastomer.

11. The method of claim 1, wherein the water-in-oil emulsion comprises from about 1% to about 5% of a silicone elastomer.

12. The method of claim 1, wherein the water-in-oil emulsion comprises from about 3% to about 5% of a silicone elastomer.

13. The method of claim 1, wherein the water-in-oil emulsion comprises a polyether-modified crosslinked siloxane.

14. The method of claim 1, wherein the water-in oil emulsion has a shear storage modulus of at least about 80 Pascals.

15. The method of claim 1, wherein the water-in oil emulsion has a shear storage modulus from about from about 80 Pa to about 650 Pa.

16. The method of claim 1, wherein the water-in oil emulsion has water phase content from about 40% to about 65% by weight.

17. The method of claim 1, wherein the water-in oil emulsion has water phase content from about 45% to about 55% by weight.

18. The method of claim 1, wherein the water-in oil emulsion is a single water-in-oil emulsion.

19. A water-in-oil emulsion, comprising:
a water phase emulsified in a continuous oil phase; and
a galvanic particulate, wherein the oil-in water emulsion has a yield stress of of at least about 20 Pa.

20. The water-in-oil emulsion of claim 19, wherein the water-in oil emulsion has a yield stress from about from about 20 Pa to about 200 Pa.

21. The water-in-oil emulsion of claim 19, wherein the water-in oil emulsion has a yield stress from about from about 20 Pa to about 100 Pa.

22. The water-in-oil emulsion of claim 19, further comprising an oil-phase rheology modifier selected from a group consisting of a silicone elastomer, a wax, a hydrophobically-modified clay, and combinations thereof.

23. The water-in-oil emulsion of claim 19, further comprising from about 3% to about 15% by weight of an oil-phase rheology modifier selected from a group consisting of a silicone elastomer, a wax, a hydrophobically-modified clay, and combinations thereof.

24. The water-in-oil emulsion of claim 19, wherein the water-in-oil emulsion comprises from about 3% to about 10% of an oil-phase rheology modifier selected from a group consisting of a silicone elastomer, a wax, a hydrophobically-modified clay, and combinations thereof.

25. The water-in-oil emulsion of claim 19, wherein the galvanic particulate is a zinc-copper particulate.

26. The water-in-oil emulsion of claim 19, wherein the emulsion comprises a silicone elastomer.

27. The water-in-oil emulsion of claim 19, wherein the emulsion comprises from about 0.25% to about 5% of a silicone elastomer.

28. The water-in-oil emulsion of claim 19, wherein the emulsion comprises from about 1% to about 5% of a silicone elastomer.

29. The water-in-oil emulsion of claim 19, wherein the emulsion comprises from about 3% to about 5% of a silicone elastomer.

30. The water-in-oil emulsion of claim 19, wherein the emulsion comprises a polyether-modified crosslinked siloxane.

31. The water-in-oil emulsion of claim 19, wherein the water-in oil emulsion has a shear storage modulus of at least about 80 Pa.

32. The water-in-oil emulsion of claim 19, wherein the water-in oil emulsion has a shear storage modulus from about from about 80 Pa to about 650 Pa.

33. The water-in-oil emulsion of claim 19, wherein the water-in oil emulsion has water phase content from about 40% to about 65% by weight.

34. The water-in-oil emulsion of claim 19, wherein the water-in oil emulsion has water phase content from about 45% to about 55% by weight.

35. The water-in-oil emulsion of claim 19, wherein the water-in oil emulsion is a single water-in-oil emulsion.
